(19) United States
(12) Patent Application Publication
(10) Pub. No.: US 2025/0009265 A1
(43) Pub. Date: Jan. 9, 2025

(54) BLOOD CIRCULATION ENHANCEMENT BRACELET

(71) Applicant: Marc Kopoian, Scottsdale, AZ (US)

(72) Inventor: Marc Kopoian, Scottsdale, AZ (US)

(21) Appl. No.: 18/219,586

(22) Filed: Jul. 7, 2023

(51) Int. Cl. A61B 5/15

(52) U.S. Cl. CPC A61B 5/150076

(57) ABSTRACT

A blood circulation enhancement bracelet improves the efficacy and ease of blood draws. A polypropylene bracelet with an adhesive closure bears a placard on one side and a sodium acetate heating pack on the opposite side. The placard bears a soothing aesthetic side which may distract and comfort the patient. The heat pack, when activated, may be placed against the inside of the patient's arm, and more particularly inside of the elbow joint. Warmth applied to the inside of the arm assists in dilating the veins in this area, making it easier for a medical professional to find and access the veins. In this way, multiple painful attempts to draw from the veins may be avoided.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### TECHNICAL FIELD

The present invention relates generally to the field of medical devices of existing art and more specifically relates to blood circulation enhancers.

### RELATED ART

Blood draws are an unpleasant experience both for patients and medical professionals. Generally, patients experience pain and trepidation while having a needle applied to draw blood. Frequently, the psychological discomfort is greater than the physical discomfort. Medical professionals may also be frustrated by the difficulty of conducting blood draws, especially with patients who have small and hard-to-find blood veins. A solution which improves ease of drawing blood both for patients and nurses, and which also address both the physiological and psychological discomforts of blood draws, is desired.

Various attempts have been made to solve problems found in eyeglass protection and storage art. Among these are found in: U.S. Patent and Publication Nos. 2016/0135254, 2015/0182376, and 10,172,762. This prior art is representative of medical devices for warming the body and improving circulation. However, these prior art solutions are expensive, complicated, and lack psychological means of improving blood circulation. Accordingly, there is still perceived a need for a superior solution.

### SUMMARY OF THE INVENTION

The present invention advantageously fills the aforementioned deficiencies by providing a novel blood circulation enhancement bracelet. The present invention is superior to other systems in that it effectively provides both physiological and psychological means for improving a patient's blood circulation.

A blood circulation enhancement bracelet improves the efficacy and ease of blood draws. A polypropylene bracelet with an adhesive closure bears a placard on one side and a sodium acetate heating pack on the opposite side. The placard bears a soothing aesthetic side which may distract and comfort the patient. The heat pack, when activated, may be placed against the inside of the patient's inside of the arm. Warmth applied to the inside of the arm assist in dilating the veins in this area, making it easier for a medical professional to find and access the veins. In this way, multiple painful attempts to draw from the veins may be avoided.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a blood circulation enhancement bracelet, constructed and operative according to the teachings of the present disclosure.

FIG. 1 is a top perspective view of the blood circulation enhancement bracelet, according to an embodiment of the disclosure.

FIG. 2 is a bottom perspective view of the blood circulation enhancement bracelet of FIG. 1, according to an embodiment of the present disclosure.

FIG. 3 is a perspective view of the blood circulation enhancement bracelet of FIG. 1 in an in-use condition, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to warming medical apparel and more particularly to a blood circulation enhancement bracelet as used to improve the physiological and psychological enhancement of a patient's blood circulation.

Generally, the blood circulation enhancement device is a bracelet which specifically targets the blood vessels passing through a wearer's inside of the arm in order to warm, dilate, and improve circulation in this part of the patient's body. A warming device may include a hermetically sealed container, such as a flexible plastic bag, containing a sodium acetate solution and a metal catalyst suspended within the solution. Preferably, the metal catalyst is a stainless steel disk which may be bent to release tiny metal particles into the solution. When the catalyst is activated in this way, the sodium acetate reacts with the metal particles, triggering a chemical reaction to crystallize the sodium acetate, releasing heat in the process.

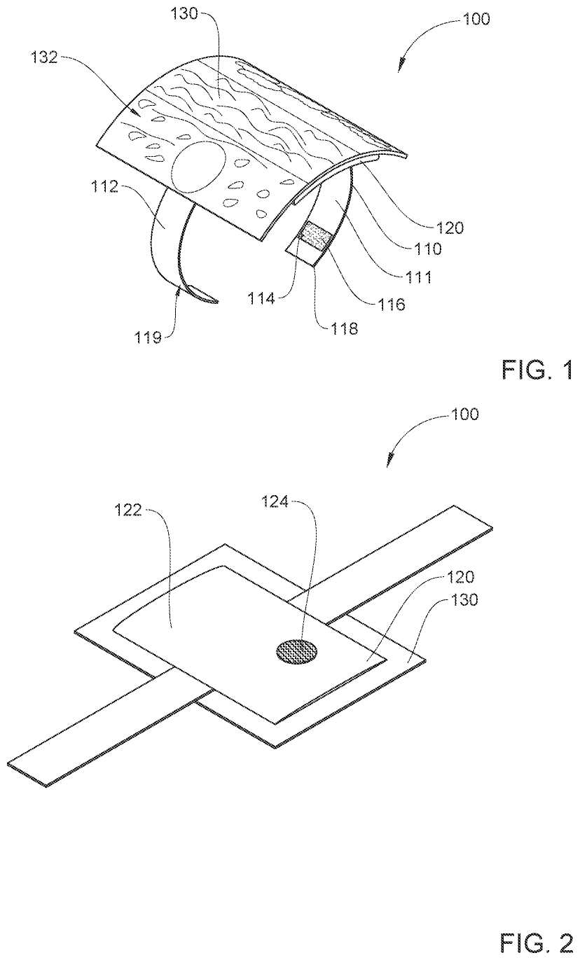

Referring now more specifically to the drawings by numerals of reference, there is shown in FIGS. 1-3, various views of a blood circulation enhancement bracelet 100.

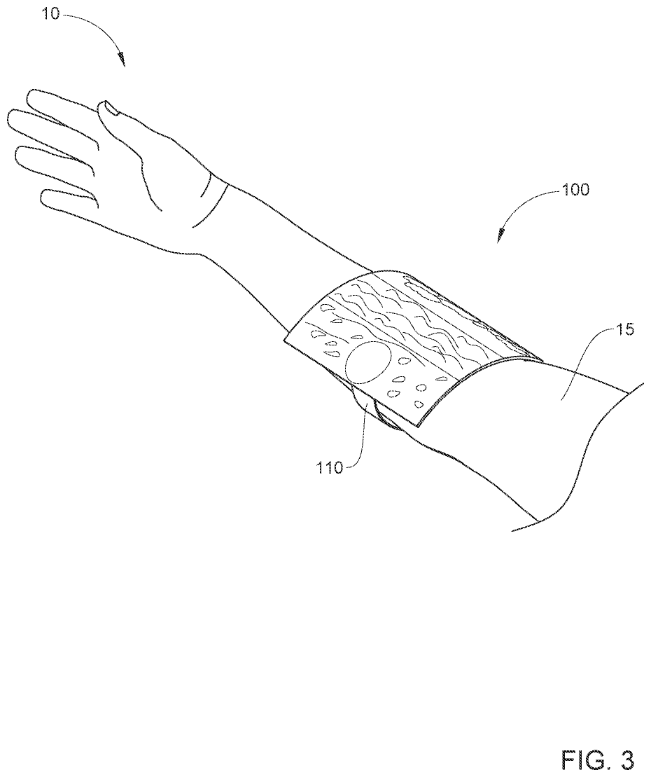

FIG. 1 shows a blood circulation enhancement bracelet according to an embodiment of the present disclosure. Here, blood circulation enhancement bracelet 100 may be beneficial for use by a user 10 (FIG. 3) to improve circulation and dilation of blood vessels at the inside of the arm 15 (FIG. 3) of user 10 (FIG. 3). As illustrated, blood circulation enhancement bracelet 100 may include a flat polypropylene band 110, a flat placard 130, and a warming device 120. It should be understood that these elements described as “flat” are flat as manufactured, being planar components having two opposite sides. Once assembled and/or worn, these components may be flexible enough to adopt a curvature (as in the case of flat polypropylene band 110, forming a circle to be worn as a bracelet).

A bracelet portion may include or consist of flat polypropylene band 110. Flat polypropylene band 110 may be characterized first side 112 and second side 111, forming opposites side of the band; these may become an outside and inside respectively when the flat polypropylene band is circled into a bracelet. Flat placard 130 may be affixed to first side 112 of flat polypropylene band 110. Warming device 120 may be affixed to second side 111 of flat polypropylene band 110, such that flat polypropylene band 110 is sandwiched between warming device 120 and flat placard 130. These elements may be adhered or otherwise fastened together. Flat placard 130 may bear a soothing design, such as a photograph of a shoreline. Flat polypropylene band 110 further comprises first end 118 and second end 119. Adhesive 114 may be applied to first end 118 of flat polypropylene band 110, and may be covered and protected by peelable cover 116. Preferably, adhesive 114 is a clear adhesive with a cover seal. In use, peelable cover 116 may be removed, flat polypropylene band 110 may encircle the arm such that warming device 120 is in contact with inside of the arm 15 (FIG. 3), and first end 118 may be pressed to second end 119, allowing first end 118 and second end 119 to be adhered together. More particularly, it is envisioned that warming device 120 be placed inside the crook of the elbow. It should be noted that the inside of the arm is seen as an ideal location for contact with warming device 120 due to the high density of blood vessels, the commonality of this area as a site for blood draws, and the ease of placement.

FIG. 2 shows blood circulation enhancement bracelet 100 of FIG. 1, according to an embodiment of the present disclosure. Warming device 120 may be affixed to second side 111 of flat polypropylene band 110 (FIG. 1) as shown. Warming device 120 faces an opposite direction from flat polypropylene band 110 (FIG. 1) as flat placard 130. Preferably, flat placard 130 is larger than warming device 120, such that warming device 120 is not visible to a user 10 (FIG. 3) when blood circulation enhancement bracelet 100 is worn. Warming device 120 may include flexible plastic container 126, solution 122, and metal element 124 suspended within solution 122. Solution 122 may be contained within flexible plastic container 126. Solution 122 may include sodium acetate and a fluid. Preferably, the fluid is water. Flexible plastic container 126 may include butene or another suitable polymer, and should be hermetically sealed to prevent escape of solution 122.

FIG. 3 is a perspective view of blood circulation enhancement bracelet 100 of FIG. 1 in an in-use condition, according to an embodiment of the present disclosure. As shown, blood circulation enhancement bracelet 100 may be worn by a user 10 to improve circulation and dilation of blood vessels at the inside of the arm 15 of user 10. Flat polypropylene band 110 encircles the arm, and flat placard 130 (FIG. 1) faces outward from the inside of the elbow. The warming device 120 (FIG. 1) may be placed inside of the arm 15 to warm blood vessels.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A blood circulation enhancement bracelet comprising:
a bracelet comprising a flat band, the flat band comprising a first side and a second side;
a flat placard affixed to the first side of the flat band; and
a warming device affixed to the second side of the flat band, the warming device comprising
a flexible plastic container,
a solution contained within the flexible plastic container, the solution comprising sodium acetate; and
a metal element suspended within the solution.

2. The blood circulation enhancement bracelet of claim 1, wherein the flat band further comprises a first end and a second end;
an adhesive is applied to the first end of the flat band; and
the adhesive is covered and protected by a peelable cover.

3. The blood circulation enhancement bracelet of claim 1, wherein the flexible plastic container comprises butene.

4. The blood circulation enhancement bracelet of claim 1, wherein the flexible plastic container is hermetically sealed.

5. The blood circulation enhancement bracelet of claim 1, wherein the flat placard comprises an image.

6. The blood circulation enhancement bracelet of claim 5, wherein the image is a landscape.

7. The blood circulation enhancement bracelet of claim 5, wherein the image is printed on the flat placard.

8. The blood circulation enhancement bracelet of claim 5, wherein the image is laminated to the flat placard.

8. The blood circulation enhancement bracelet of claim 1, wherein the flat band is constructed of polypropylene.
